(19) United States
(12) Patent Application Publication
(10) Pub. No.: US 2019/0103178 A1
(43) Pub. Date: Apr. 4, 2019

(54) METHOD AND DEVICE FOR PRODUCING AN ELECTRONIC HEALTH RECORD FROM PAPER

(71) Applicant: Pratap Kumar, Nairobi (KE)

(72) Inventor: Pratap Kumar, Nairobi (KE)

(21) Appl. No.: 16/208,594

(22) Filed: Dec. 4, 2018

Related U.S. Application Data
(63) Continuation of application No. PCT/IB2017/053205, filed on May 31, 2017.

(30) Foreign Application Priority Data
Jun. 2, 2016 (KE) KE/UM/2016/00657

(51) Int. Cl. G16H 15/00; G06K 9/00; G16H 10/60

(52) U.S. Cl. CPC G16H 15/00; G06K 9/00449; G16H 10/60; G06K 9/00463; G06K 9/00469

(57) ABSTRACT

Although mobile technology has great potential for assisting a variety of activities in low-resource settings, there have been few successes at scale. Herein is provided an innovation that improves access and quality while considering the costs of healthcare. The innovation efficiently extracts digital information from paper-based records using low-cost and locally produced rubber stamps to improve adherence to clinical practice guidelines. By bringing down the costs of clinical audit, this innovation offers the potential for clinics in resource-limited settings to deliver high-quality care.

### CROSS REFERENCE TO RELATED APPLICATIONS

This application claims priority to PCT application Ser. No. PCT/IB2017/053205, filed 31 May 2017, which claims priority to Kenyan provisional application serial number KE/UM/2016/00657, filed 2nd Jun. 2016, the contents of which are incorporated by reference in their entirety.

### INTRODUCTION

E-Health, a broad term inclusive of mobile health (m-Health) that encompasses electronic-supported processes and communication in healthcare, is making forays in areas as diverse as medical recordkeeping, clinical decision support and coordination of care in almost every country and setting. Coupled with mobile connectivity, e-Health has been trumpeted as a means of revolutionising access to healthcare. It is broadly expected that such technology will positively impact access and quality of healthcare services in low- and middle-income countries (LMICs).

E-Health not yet engendered the ‘revolution’ in access to and quality of medical care in LMICs. Some reasons include traditional challenges such as the severe shortfall of human resources that limit any initiative to increase access to care. Others include ‘human’ issues that limit the buy-in and use of new technologies by their stakeholders. Variously referred to as ‘human centred design’ or ‘process innovation’, attention to existing workflows, incentive structures, participation of users at multiple levels of implementation, and broader links into the health system are all important issues that can determine the success of e-Health interventions. Even if a pilot is considered ‘successful’, small-scale implementations often lack programmatic evidence to inform the scale-up of e-Health interventions.

Over the last two decades, e-Health systems such as Electronic Medical Records (EMR), Picture Archiving and Communication Systems (PACS) and Patient Administration Systems (PAS) have been widely adopted in high-income countries. This adoption has been achieved at a high cost, and different drivers of adoption, from medical coding and billing to participating in government programs, have resulted in systems that are often difficult to use at the point of care and limited in their ability to share clinical information across facilities. Increasing emphasis is therefore being placed on issues of interoperability and clinical usability.

To address the problems with sharing data and allowing e-Health systems to communicate with each other, a process of international standardisation has emerged. However, much work is still needed to ensure seamless integration of systems and secure sharing of health data. By using internationally agreed standards, the global marketplace for e-Health systems can grow with less friction and benefit from pooling expertise from different countries. The market in LMICs, where e-Health systems are just starting to be implemented at scale, could especially benefit from such efforts.

### BRIEF DESCRIPTION OF THE INVENTION

In an aspect is a method for creating an electronic health record from paper, the method comprising: forming an imprint on a medium with a stamp, the imprint comprising one or more reference elements and one or more data fields; entering data into at least one data field of the imprint; and forming a digital image of the imprint and extracting data from the digital image. In embodiments:

the medium is paper;

the stamp is a rubberized stamp;

the imprint is an ink imprint;

the imprint comprises reference elements at corners of the imprint, the reference elements providing edge and orientation information;

the imprint comprises 3 or 4 reference elements;

the entering data comprises forming machine-readable marks in the data field or fields;

the digital image is formed using a digital camera;

the extracting data comprises digitally analysing the digital image with software capable of reading the entered data;

further comprising adding the extracted data to a database of extracted data;

the forming the imprint comprises coating the stamp with ink and pressing the stamp on the substrate to transfer the ink from the stamp to the substrate; and

further comprising forming an electronic health record from the extracted data.

In an aspect is a device for carrying out the method as above. In embodiments the device comprises a rubberized stamp and a handle. In embodiments the rubberized stamp comprises at least one reference element and a plurality of standardized data fields such that, when coated with ink and pressed against a medium, the rubberized stamp creates an imprint on the medium, the imprint comprising the at least one reference element and the plurality of standardized data fields.

In an aspect is a method for creating an electronic health record, the method comprising: analysing a digital image of a processed stamp impression to extract standardized data from the impression, and forming an electronic health record from the extracted standardized data. In embodiments the processed stamp impression comprises a plurality of standardized data fields and at least one reference element.

In an aspect is a method comprising: preparing a form relating to an entity, wherein said form comprises machine readable data fields identifying said entity, machine readable selection items relating to data to be captured by said form, and at least one reference element; providing said form on a medium that is capable of being marked; after markings have been placed on said form by a user who interacted with said entity, scanning said form to produce an image file; analyzing said image file to identify whether user added markings are present on said machine readable selection items; obtaining electronically storable data relating to said entity based on said user added markings on said machine readable selection items; and outputting said electronically storable data relating to said entity.

### BRIEF DESCRIPTION OF THE DRAWINGS

FIG. 1: a first example of a full-scale illustration of a template for managing a health condition.

FIG. 2: a second example of a full-scale illustration of a template for managing a heath condition.

FIG. 3: a third example of a full-scale illustration of a template for managing a health condition.

### DETAILED DESCRIPTION OF VARIOUS EMBODIMENTS

To be effective, clinical practice guidelines (CPGs) need to be: a) developed or adapted in an inclusive manner with direct input from the medical staff who will be implementing them, b) included into existing workflows without requiring significant additional effort or cost, and c) paired with systems to measure, evaluate, feedback and reward evidence-based practice, thus motivating providers to deliver high-quality clinical care. While the impact of audits and feedback in LMIC healthcare systems can be high, one factor limiting the use of such methods is the high cost of performing audits, particularly in the absence of EMRs. The alternatives to clinical audits for collecting data on quality of clinical care in LMICs are also expensive, involving detailed interviews and observations, or standardised patients.

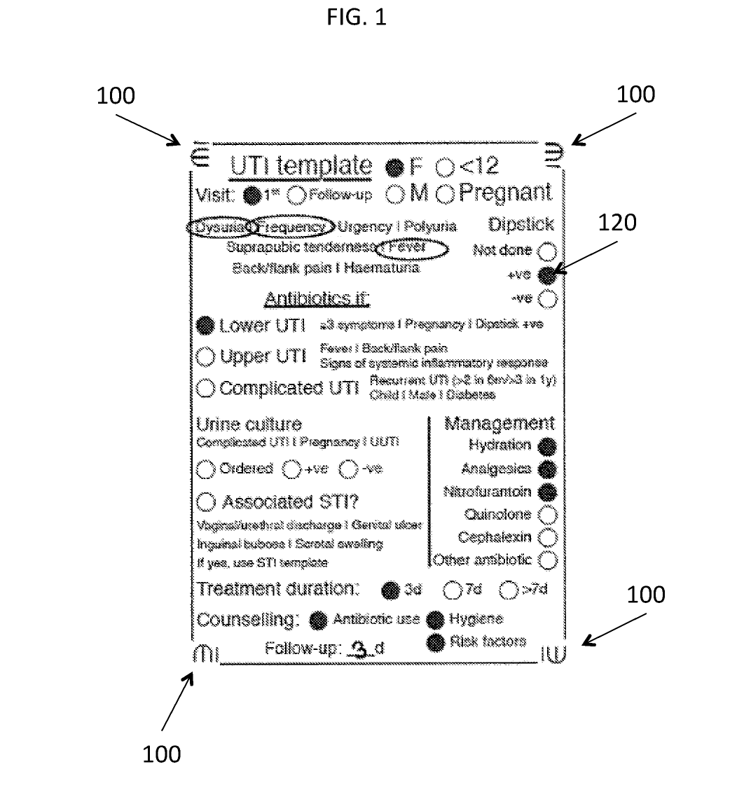

A solution to some of these challenges of measuring and improving quality of clinical care is the use of checklists, as widely demonstrated in surgical settings. An extension of this concept involves the use of rubber stamps to place checklists to support the diagnosis and management of various clinical conditions into paper case sheets for use during patient encounters. The invention has prototyped rubber stamp CPG templates (RST) that are condition-specific (e.g. urinary tract infection, hypertension, etc.), and incorporate important elements of relevant CPGs, diagnostic differentials and treatment options while accounting for the limitations of a particular clinic or clinical setting. The RSTs (FIG. 1) function both as a ‘checklist’, reminding providers of often-missed care (e.g. screening for sexually transmitted infections in cases presenting as urinary tract infections), and a means to record important clinical information (e.g. antibiotic used and justification for use). RSTs are designed for easy digitisation using a mobile phone camera, rapidly capturing key clinical data in settings with limited resources. Results from RST use show promising reductions in antibiotic prescription rates for common infections and other metrics of quality of clinical care, as well as enthusiastic support from providers using the tools.

The device of the invention aims to deliver and drive the demand for high-quality health services by a) improving quality of care at the facility level, and b) increasing data available to health providers on patient level care.

The invention provides rubber stamp CPG templates (RST) that can be placed on a paper case sheet, and the resulting impression can be easily filled during patient encounters. In embodiments each RST (˜7×10 cm or any other convenient size) is condition specific (e.g. pre-eclampsia, diarrhoeas), incorporating guidelines, diagnostic differentials and treatment recommendations. Data entered in the RST impression can be easily digitised using a mobile phone camera or other camera. Digitization is aided by elements of the impression—e.g., corner locators, orientation reference elements, and the like. In embodiments, case sheets are designed as digitisable clinical encounter forms (dCEF) in a similar manner to the RSTs described herein, such that key demographic data, history, diagnoses, tests and treatment can be extracted from paper and fed into an EMR database. Such tools will not only enable EMR penetration into rural sites, but also improve quality of care delivered at multiple levels of the health system.

Standard EMRs are expensive, difficult to use and cumbersome to implement and maintain. The devices according to the invention attempt to incentivise thorough documentation on paper, and simultaneously enable data-driven management of clinical care. This includes improve adherence to guidelines—The design of both dCEFs and RSTs are founded on improving adherence to CPGs. Besides important demographic information, data entered in these formats will focus on information that will improve quality of care (e.g. early detection of high-risk pregnancies, justification of antibiotic use).

The devices according to the invention are tools that will enable near real-time monitoring and analysis of patient care. For example, case data is routinely entered on a daily basis using these tools, but higher frequency is possible). Facility performance on management of key health conditions can be fed back to the providers to identify and correct gaps (e.g. equipment failures, stock-outs, lack of provider knowledge).

The use of paper to interface with EMRs can highlight the benefits of EMRs while simplifying their use. This can aid the wider use of EMRs in the future.

The devices according to the invention aim to improve the quality of management of common health conditions. This includes early detection of high-risk cases, better treatment through adherence of guidelines, and reduced MNH mortality and morbidity.

The devices according to the invention allow benefits that include significant reduction in antibiotic use and improved diagnosis and management of UTIs and STIs (key steps to reducing risk of pre-term labour). The devices and methods marry the use of RSTs with EMR technology and is a unique, innovative solution to healthcare in the developing world.

The devices according to the invention use a creative approach to improving quality of clinical care, combining simple, cheap front-end technologies (paper forms, rubber stamp, and mobile phone camera) with sophisticated back-end technologies (automated image recognition & analysis, current e-Health standards, and commonly used EMR databases). RSTs allow cost-effective incorporation of checklists and complex evidence-based care protocols into a patient-facing scenario.

Templates are tailored to be context specific with important elements of the CPG matched to each clinic's constraints and performance. Such tailoring of CPGs is recommended by all guideline developers but is rarely done. CPG stamps can be tested and revised, and quickly scaled and implemented at other public sector facilities with simple clinical and technological support. Further modification is inexpensive, involving only a change in the rubber engraving (minimal wastage of paper or complicated software upgrades). Other than the rubber engraving portion, the remainder of each stamp can be reused by gluing or otherwise affixing new engravings as desired.

The devices according to the invention require minimal changes to health facility charting. Existing paper case sheets can be modified into digitisable clinical encounter forms (dCEF). Placing and filling the appropriate RST into the case sheet takes an additional 30 s in the patient encounter. In embodiments optical ‘bubble sheet’ format and other techniques are employed to efficiently extract data from paper documentation, effectively combining a simple user interface (paper & pen) with EMR databases.

In embodiments, data from RSTs alone provide detailed, de-identified, facility-level quality scores for selected health conditions. When combined with demographic data from the dCEF, the data becomes a full-fledged electronic patient record allowing continuity of care, referral management and other benefits of an EMR.

The devices according to the invention provide a convenient bridge to healthcare providers and managers into the use of EMRs without significant investment into user interface technology (typically on tablets or laptop computers) and training (a significant hurdle for optimal uptake of EMRs).

The devices according to the invention have the potential to improve outcomes by promoting adherence to evidence-based CPGs and using tools to measure/monitor such adherence. Specific outputs include close follow-up of high-risk pregnancies or other conditions, early detection/management of disease and complications, and targeted referral.

Examples of templates include any of the following pregnancy-related conditions, and others conditions and health procedures (e.g., routine medical checks, etc.) as desired.

Hypertensive disorders—the devices according to the invention provide templates that will promote early detection and care of hypertension, e.g., in pregnancy. This includes decision support for on-site treatment versus referral, and appropriate care that can be delivered at the primary care level to assure safe delivery.

Ante-partum haemorrhage—APH occurs in 2-6% of pregnancies and is a leading cause for maternal deaths. Pre-existing anaemia is common and increases the risk of a fatal outcome. Focussed antenatal care is the most cost-effective strategy for preventing these deaths. The devices according to the invention include templates that will promote early detection of risk factors, crucial for counseling mothers on the appropriate place of delivery, and danger signs/symptoms that should prompt them to seek assistance.

Newborn sepsis—the devices according to the invention aim to prevent neonatal sepsis by improving detection and standardised management of serious infections at community—and dispensary/health centre-level.

The devices according to the invention will not only measure knowledge and practices in healthcare industry, but also incorporate these data into an EMR database to identify and correct gaps. The use of EMRs is expected to improve quality of care by increasing data available for analysis and decision making.

The devices and methods of the invention are tools that a) improve routine clinical documentation in patient facing scenarios, b) increase adherence to clinical practice guidelines, c) allow for rapid digitization and analysis using mobile phone cameras, and d) facilitate the use of quality improvement methodologies to address gaps in practice. The use of these tools can increase the ability for healthcare providers in low-resource settings to identify, triage, manage and refer pregnant women and newborns at risk.

Traditional paper documentation of clinical care, though straightforward, is not easily compatible with evidence-based clinical quality improvement. Clinics often avoid audits of clinical practice that can generate this evidence, as they require chart reviews or observations that are tedious, error-prone, and require significant resources. Electronic medical records (EMR) on the other hand are expensive, often difficult to use in patient-facing scenarios, and cumbersome to implement and maintain. The devices and methods of the invention are a unique combination of paper-based documentation tools and EMRs. Specially designed rubber stamp templates (RST) allow easy and thorough documentation at the point of care, and rapid digitization and analysis using a mobile phone camera.

Templates are developed using human-centered design and participatory methods, and incorporate important elements of clinical practice guidelines (CPG)/best-practice protocols, diagnostic differentials and treatment recommendations. The rubber stamp is manufactured in the normal method or any appropriate method (known or later developed) for producing such stamps.

Appropriate templates are placed as images on paper case sheets using a rubber stamp, and easily filled during patient encounters. For example, each RST addresses a different point in the continuum of maternal and newborn healthcare (MNH), focusing specifically on gaps in the quality of clinical care. Data entered in the RST (or in modified versions of common charts such as partograms) can be easily digitized using a mobile phone camera and computer vision algorithms to feed into an EMR database. These tools not only improve clinical documentation, but also enable the facility to retain a copy of the patient record, facilitating data-driven management of clinical services and evidence-based quality improvement initiatives.

Data from the tools herein can be used to drive improvements in quality of care across the healthcare continuum by, for example, 1) identifying gaps in diagnosis, treatment and referral which can be addressed through training, 2) feeding back clinical quality data using dashboards to clinical and management teams for facility-level performance evaluation, 3) using data to inform Plan-Do-Check-Act (PDCA) cycles to rapidly test quality improvement interventions, and 4) allowing for iteration of RSTs and other tools based on use and staff feedback to ensure the tools are customized and contextualized to each facility while still ensuring fidelity with the CPGs/algorithms.

Additional features of RST design include: a) Concomitant narrative documentation: Being limited in size (e.g., about 7×10 cm, or any other size as desired and convenient), RSTs do not replace the need or the ability to provide narrative documentation. Similarly, multiple comorbidities can be easily documented on a single paper sheet. b) Complement detailed CPGs & algorithms: Stamp-based templates incorporate specific aspects of the guidelines without necessarily transforming an entire guideline into a decision algorithm, thus potentially saving time and effort and highlighting key actions. c) Integrate clinical decision support and documentation: RSTs combine clinical aides like checklists and diagnostic/treatment criteria with simple data entry methods. Such ‘dual use’ drives support from both clinicians and managers. d) Focus on specific indicators/outcomes. Such goal-driven design allows generic tools like rubber stamps and mobile phones to tackle highly specific problems.

Rubber stamps according to the disclosure can be used even when the patient record is a patient-held notebook. This is especially relevant to clinical practice in low-resource settings like Kenya where, in the example of pregnancy, mothers carry their maternity case records with them and clinics rarely maintain patient records at the facility. By avoiding the need to implement laptop/tablet computers at the point-of-care, the methods and devices of the invention can highlight the benefits of EMRs while simplifying their use, thus aiding the wider use of digital systems. This will not only enable EMR penetration into rural sites, but also improve quality of care delivered at multiple levels of the health system.

A clinic using the methods and devices of the invention requires one or more of the RST, a device for imaging the physical records produced with the RST (e.g., a cell phone camera or other camera), a computer (along with suitable software) for receiving, storing, and analyzing the images, and personnel for operating the equipment. An optional internet connection or other network connection allows the clinic to share such data, store/analyze such data on a cloud platform, receive software updates, etc. Modification of RSTs is cheap, involving only a change in the rubber engraving, the skills for which are abundant locally.

Any suitable materials can be used to prepare the devices of the invention. Rubberized stamps typically use a standard petroleum by-product for the rubber material but any other suitable material can be used (e.g., silicon-based, etc.). The handle and backing/substrate for the rubber stamp can be of any material and/or any suitable shape. Examples include wood, plastic, metal, and combinations thereof.

In an aspect is a method for creating an electronic health record from paper, the method comprising: forming an imprint on a medium with a stamp, the imprint comprising one or more reference elements and one or more data fields; entering data into at least one data field of the imprint; and forming a digital image of the imprint and extracting data from the digital image.

In embodiments, the medium is paper, including any color, texture, and weight of paper.

In embodiments, the stamp is a rubberized stamp, although the stamp may be non-rubberized and formed from any suitable material including synthetic and natural materials capable of forming an imprint on the medium when ink is applied to the stamp.

In embodiments, the imprint is an ink imprint. Thus the method may further include the step of applying ink to the stamp.

In embodiments, the imprint comprises reference elements at corners of the imprint, the reference elements providing edge and orientation information. A reference element is any image that, when present in specific number, location, and orientation, enables a digital/automatic scanner device to determine the overall orientation of the imprint. The reference elements are present in the imprint and are therefore also present in the stamp. In embodiments, the reference elements may be 3 or 4 in number, located at 3 or 4 (respectively) corners of the stamp/imprint. In embodiments one or more reference elements may be located other than at a corner, such as at an edge or in the center of the stamp/imprint. In embodiments, the imprint comprises a single reference element at the centre of the imprint (and the stamp comprises the corresponding element at the corresponding location), wherein the single reference element provides sufficient orientation information. In such embodiments, the outer edges of the stamp/imprint may require delineation (e.g., a border line) for the purposes of scanning and data entry.

In embodiments, the entering data comprises forming machine-readable marks in the data field or fields. Such marks may be, for example, filled bubbles, partially filled bubbles, or “X” marks, or some other mark that is machine-readable such as by a scanning device. The step of entering data is carried out by a user (e.g., a physician, nurse, or other healthcare professional) and the user may be using any physical marking tool such as a pen, pencil, or other tool for making machine-readable marks on the paper comprising the imprint.

In embodiments, the digital image is formed using a digital camera. In embodiments, the digital image is formed using a digital scanner.

In embodiments, the extracting data comprises digitally analysing the digital image with software capable of reading the entered data. Such software may include optical character recognition (OCR) software although in embodiments OCR is not necessary because of the standardization of the data from imprint to imprint. Thus the stamp methods described herein remove the need for the software to be able to recognize elements other than the machine-readable marks described herein.

In embodiments, further comprising adding the extracted data to a database of extracted data. The software may be sufficiently arranged to be capable of processing incomplete imprints (e.g., where a physician or other user neglects to complete making data marks on the imprint).

In embodiments, the forming the imprint comprises coating the stamp with ink and pressing the stamp on the substrate to transfer the ink from the stamp to the substrate.

In embodiments, further comprising forming an electronic health record from the extracted data. In this manner, the data entered by the user in physical form (i.e., on the paper comprising the imprint) becomes digitized data in an e-health record.

In an aspect is a device for carrying out the method as above. In embodiments the device comprises a rubberized stamp and a handle.

In an aspect is a method for creating an electronic health record, the method comprising: analysing a digital image of a processed stamp impression to extract standardized data from the impression, and forming an electronic health record from the extracted standardized data. In embodiments the processed stamp impression comprises a plurality of standardized data fields and at least one reference element.

In an aspect is a method comprising: preparing a form relating to an entity, wherein said form comprises machine readable data fields identifying said entity, machine readable selection items relating to data to be captured by said form, and at least one reference element; providing said form on a medium that is capable of being marked; after markings have been placed on said form by a user who interacted with said entity, scanning said form to produce an image file; analyzing said image file to identify whether user added markings are present on said machine readable selection items; obtaining electronically storable data relating to said entity based on said user added markings on said machine readable selection items; and outputting said electronically storable data relating to said entity.

Certain aspects of the invention are shown in the attached figures. With reference to FIG. 1, there is provided an illustration of a template for managing urinary tract infections (UTI). The template is placed in the case sheet using a rubber stamp. Fiducial markings (reference elements) 100 in the corners allow for accurate digitisation and image analysis using mobile phone cameras or another scanner. The optical ‘bubble’ sheet (e.g., filled bubble 120) format allows for rapid and efficient extraction and analysis of digital data from paper-based medical records.

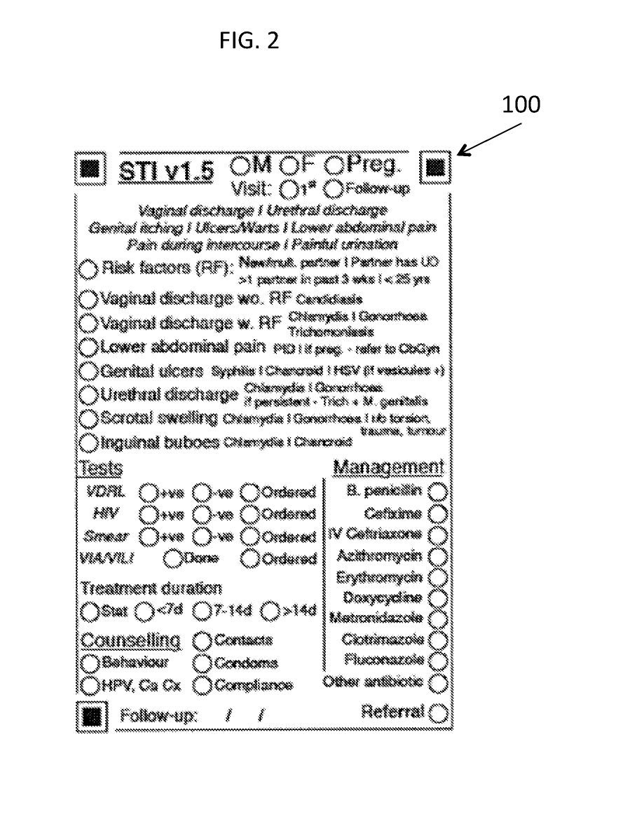

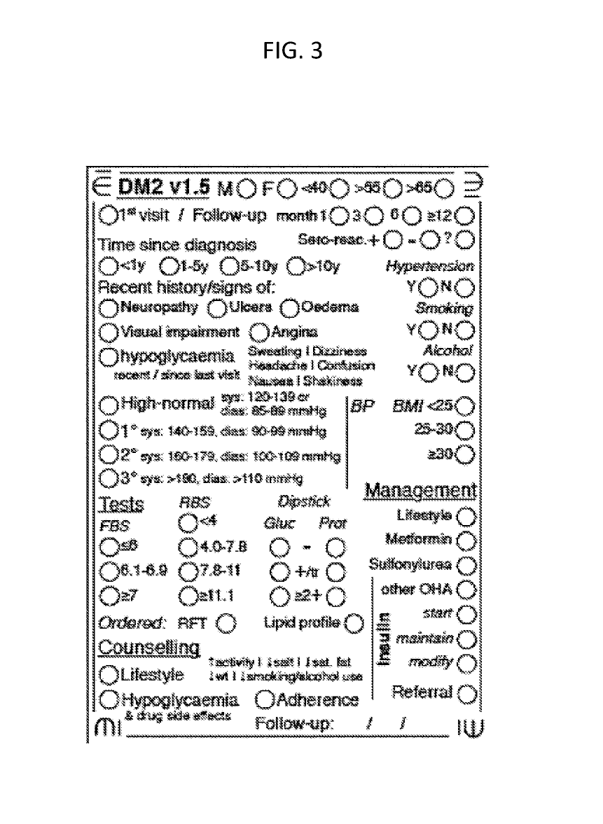

FIGS. 2 and 3 provide further examples of impressions from rubber stamps according to the invention.

It is to be understood that while the invention has been described in conjunction with examples of specific embodiments thereof, that the foregoing description are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. Any combination of the embodiments described herein is intended to be part of the invention, as if all such combinations had been laboriously set forth in this disclosure.

### EXAMPLES

Over six months a pilot was conducted involving the use of RSTs for common outpatient (nonmaternal) conditions in seven clinics in the slums of Nairobi. Outcomes include significant reduction in antibiotic use and improved diagnosis and management of UTIs and STIs (which are key steps to reducing risk of pre-term labor). For example, antibiotic usage for upper respiratory tract infections decreased from 96% to 54%, documentation of temperature increased from 58% to 87%, and usage of first-line antibiotic for urinary tract infections increased from 17% to 100%. The RSTs are designed to be highly context specific and tailored to the needs and challenges of each clinic, e.g. documenting clinic-specific protocols like follow-on phone calls or targeted SMS messaging. Such clinic-level customization improves buy-in for the use of these tools by the management and healthcare staff. The tools also decrease wastage and overcome the logistical challenges of documentation systems that are restricted to either paper or electronic methods only. Preliminary observations show that providers and clinic managers prefer a bank of rubber stamps to managing pre-printed paper templates.

1. A method for creating an electronic health record from paper, the method comprising:
forming an imprint on a medium with a stamp, the imprint comprising one or more reference elements and one or more data fields;
entering data into at least one data field of the imprint; and
forming a digital image of the imprint and extracting data from the digital image.

2. The method of claim 1, wherein the medium is paper, the stamp is a rubberized stamp, and the imprint is an ink imprint.

3. The method of claim 1, wherein the imprint comprises reference elements at corners of the imprint, the reference elements providing edge and orientation information.

4. The method of claim 1, wherein the entering data comprises forming machine-readable marks in the data field or fields.

5. The method of claim 1, wherein the extracting data comprises digitally analysing the digital image with software capable of reading the entered data.

6. The method of claim 1, further comprising adding the extracted data to a database of extracted data.

7. A device for carrying out the method of claim 1.

8. The device of claim 7, comprising a rubberized stamp and a handle.

9. The device of claim 8, wherein the rubberized stamp comprises at least one reference element and a plurality of standardized data fields such that, when coated with ink and pressed against a medium, the rubberized stamp creates an imprint on the medium, the imprint comprising the at least one reference element and the plurality of standardized data fields.

10. A method for creating an electronic health record, the method comprising: analysing a digital image of a processed stamp impression to extract standardized data from the impression, and forming an electronic health record from the extracted standardized data.

11. The method of claim 10, wherein the processed stamp impression comprises a plurality of standardized data fields and at least one reference element.

12. A method comprising:
preparing a form relating to an entity, wherein said form comprises machine readable data fields identifying said entity, machine readable selection items relating to data to be captured by said form, and at least one reference element;
providing said form on a medium that is capable of being marked;
after markings have been placed on said form by a user who interacted with said entity, scanning said form to produce an image file;
analyzing said image file to identify whether user added markings are present on said machine readable selection items;
obtaining electronically storable data relating to said entity based on said user added markings on said machine readable selection items; and
outputting said electronically storable data relating to said entity.
